(19) United States
(12) Patent Application Publication
(10) Pub. No.: US 2026/0369394 A1
(43) Pub. Date: Nov. 19, 2026

(54) SYSTEM AND METHOD FOR INCREASING EFFICIENCY OF TURNOVER TIME IN SURGICAL OPERATING ROOMS

(71) Applicant: S1 PIT CREW INC, SAN DIEGO, CA (US)

(72) Inventor: Ramin Raiszadeh, San Diego, CA (US)

(21) Appl. No.: 19/662,757

(22) Filed: Apr. 29, 2026

Related U.S. Application Data
(60) Provisional application No. 63/806,503, filed on May 15, 2025.
(60) Provisional application No. 63/817,115, filed on Jun. 3, 2025.
(60) Provisional application No. 63/846,284, filed on Jul. 18, 2025.
(60) Provisional application No. 63/974,090, filed on Feb. 2, 2026.

(51) Int. Cl. G16H 40/20; G06F 16/28; G06Q 10/0633

(52) U.S. Cl. CPC G16H 40/20; G06F 16/285; G06Q 10/0633

(57) ABSTRACT

The present disclosure relates to an application and method for increasing efficiency and consistency of turnover times in operating rooms of medical establishments. The application records and displays time splits based upon user input for time taken to complete each task individually and the turnover in its entirety. The application further allows healthcare staff to see real time data regarding the start, progression, and completion of turnovers in multiple operating rooms.

### CROSS-REFERENCE TO RELATED APPLICATIONS

This application is related to U.S. Provisional Application No. 63/974,090 filed Feb. 2, 2026, U.S. Provisional Application No. 63/846,284 filed Jul. 18, 2025, U.S. Provisional Application No. 63/817,115 filed Jun. 3, 2025, and U.S. Provisional Application No. 63/806,503 filed May 15, 2025, the contents of which are expressly incorporated herein by reference.

### STATEMENT RE: FEDERALLY SPONSORED RESEARCH/DEVELOPMENT

Not Applicable

### BACKGROUND

### 1. Technical Field

The present disclosure relates generally to the field of software systems designed to track timing of tasks and facilitate efficiency and consistency in a real-time medical setting. More particularly, the present disclosure relates to a software system including a user application for mobile devices for tracking turnover time between patients in operating rooms, including a method of implementation of the same.

### 2. Related Art

Efficiency and consistency in healthcare is sought by both patients and healthcare providers alike to provide streamlined and quality care to every single patient while allowing healthcare professionals to work predictable schedules. Yet, the fast-paced and unpredictable nature of healthcare work proves a major barrier to the streamlined output seen in other fields such as manufacturing, retail, and food service. The present disclosure serves to propose a software and method of use for promoting efficiency in healthcare settings. More specifically, the present disclosure proposes a mobile application for tracking data related to turnover times between patients in operating rooms to increase the speed and precision in which the turnover is completed. This application aims to ameliorate the two largest problems associated with turnover times in surgical settings: the length of time taken to complete a turnover and inconsistencies in the time taken to complete a turnover.

A long and/or inconsistent turnover time between patients in an operating room causes a myriad of problems for the hospital, healthcare staff, and patients alike. Long turnover times may cause the hospital to lose revenue due to inefficiency and manpower that could be used elsewhere while the turnover is occurring. The hospital must likewise deal with uncertainties in allocation of manpower and resources as well as an uncertain budget for payment of its staff due to inconsistent turnover times. The healthcare staff, specifically the surgeons, may suffer from frustration, burnout, or dissatisfaction due to the long and inconsistent waits between patients which may be exacerbated by uncertainty in shift length, causing stress not only in the staff's professional life but their personal life as well. The patients and their loved ones may likewise become frustrated due to the long waits for an operation, which may, in turn, prove costly to the individual due to longer stays in the hospital.

Still further, surgeons may deal with frustrations and inefficiencies linked to incorrect or missing equipment in the operating room. Surgeons will often provide preference cards to the healthcare staff in charge of turning over the operating room between patients, but certain preferences or equipment may not be prepared by the turnover team for a variety of reasons. Physical preference cards may be lost, not presented to each and every member of the turnover team, or presented to the team too late to ask necessary questions.

In an effort to ameliorate these drawbacks, inspiration was taken from pit crew teams in professional motorsports to create an application designed to increase efficiency of turnovers. Pit crews in Formula 1 racing, for example, are able to change tires in record time with impressive teamwork and extreme coordination. The present disclosure aims to implement this strategy of teamwork and coordination into a medical setting by providing a software and method of implementation that allows for tracking of turnover times to increase efficiency and consistency of turnover times in surgical settings.

### BRIEF SUMMARY

To solve the problem of inconsistencies in turnover times in healthcare facilities, an application for collecting and displaying time data for turnovers on multiple mobile devices is contemplated herein. In such an application, a processor communicates with and collects data from multiple mobile devices and displays real-time data as to the status of the turnover on multiple mobile devices. The processor receives user input from a mobile device, indicating to start a stopwatch time sequence. Additional user input is categorized into specific tasks and is assigned a completion time based upon when the user initiates the input. Said completion time of the task is then displayed on multiple mobile devices. For instance, a user input indicating that a first task is complete assigns that user input a first timestamp, the first timestamp is assigned as the completion time of that first task and is displayed as such on multiple mobile devices. A user input indicating that a second task is complete likewise assigns that user input with a second timestamp and the second timestamp is assigned as the completion time of the second task and is displayed as such. A user input indicating completion of the turnover will stop the stopwatch time sequence and create an ending timestamp. That ending timestamp is assigned as the time taken to complete the turnover in its entirety and is displayed as such on multiple mobile devices.

The application may also include special categorized user inputs. One such special categorized user input prompts a countdown timer to begin, notifying the user that the countdown timer is depleted and that the next task may be started. Another special categorized user input displays a popup notification, requiring user input to dismiss from the mobile device display. A final special categorized user input displays a popup notification, requiring the user to input either yes or no to a question proposed in the popup to dismiss the notification.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a schematic overview of a turnover management system;

FIG. 2 is a block diagram of an example user device of the turnover management system having a user application;

FIG. 3 is a block diagram of a storage management tool of the turnover management system;

FIG. 4 is a block diagram of an example application management tool of the turnover management system having a master program;

FIG. 5 is a screenshot of a user interface of the user application depicting an example of a real-time turnover schedule showing a completed turnover and a turnover in progress;

FIG. 6 is a screenshot of a user interface of the user application depicting an example of a real-time turnover schedule for an operating room, showing a turnover in progress;

FIG. 7 is a screenshot of a user interface of the user application depicting an example of a real-time turnover schedule for a particular user or role;

FIG. 8 is a screenshot of a user interface of the user application depicting an example of a role selection prompt;

FIG. 9A is a screenshot of a user interface of the user application depicting an example of a first stage of a turnover workflow;

FIG. 9B is a screenshot of a user interface of the user application depicting an example of a second stage of a turnover workflow;

FIG. 9C is a screenshot of a user interface of the user application depicting an example of a third stage of a turnover workflow;

FIG. 10 is a screenshot of a user interface of the user application depicting an example of a turnover workflow while the turnover is in progress;

FIG. 11 is a screenshot of a user interface of the user application depicting an exemplary a data overview of a completed turnover;

FIG. 12 is an image depicting an example of a preference card;

FIG. 13 is a flowchart depicting a first example of a non-standard turnover workflow;

FIG. 14 is a flowchart depicting a second example of a non-standard turnover workflow; and

FIG. 15 is a high-level block diagram of an exemplary apparatus that may be used to implement systems and methods described herein.

### DETAILED DESCRIPTION

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways. Also, it is to be understood that the phraseology and terminology used herein is for the purpose of description and should not be regarded as limiting.

The detailed description set forth below in connection with the appended drawings is intended as a description of certain embodiments of an application and/or system for increasing efficiency of turnover times in surgical operating rooms and is not intended to represent the only forms that may be developed or utilized, nor are the described methods the only methods that could be employed. The description sets forth the various structure and/or functions in connection with the illustrated embodiments, but it is to be understood, however, that the same or equivalent structure and/or functions may be accomplished by different embodiments that are also intended to be encompasses within the scope of the present disclosure. It is further understood that the use of relational terms such as first and second, and the like are solely to distinguish one entity from another without necessarily requiring or implying any actual such relationship or order between such entities.

It should be noted that any language directed to a computer should be read to include any suitable combination of computing devices, including servers, interfaces, systems, databases, agents, peers, engines, controllers, or other types of computing devices operating individually or collectively. One should appreciate the computing devices comprise a processor configured to execute software instructions stored on a tangible, non-transitory computer readable storage medium (e.g., hard drive, solid state drive, RAM, flash, ROM, etc.). The software instructions preferably configure the computing device to provide the roles, responsibilities, or other functionality as discussed below with respect to the disclosed apparatus. Further, the disclosed technologies can be embodied as a computer program product that includes a non-transitory computer readable medium storing the software instructions that causes a processor to execute the disclosed steps. In especially preferred embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges preferably are conducted over a packet-switched network, the Internet, LAN, WAN, VPN, or other type of packet switched network.

The following discussion provides several example embodiments of the present disclosure. Although each embodiment represents a single combination of disclosed elements, the present disclosure is considered to include all possible combinations of the disclosed elements. Thus if one embodiment comprises elements A, B, and C, and a second embodiment comprises elements B and D, then the present disclosure is also considered to include other remaining combinations of A, B, C, or D, even if not explicitly disclosed.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously. Within the context of a networking environment, the terms “coupled to” and “coupled with” are also used euphemistically to describe two or more networked computing devices that are communicatively connected with each other over a network, possibly via one or more intermediary devices.

It should be apparent to those skilled in the art that many more modifications besides those already described are possible without departing from the subject matter disclosed herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refer to at least one of something selected from the group consisting of A, B, C . . . and N, the text should be interpreted as requiring only one element from the group, not A plus N, or B plus N, etc.

Bearing the foregoing in mind, the present disclosure relates to a turnover management system for tracking turnover times between patients in operating rooms in an effort to improve efficiency and consistency of said turnover times and a method of implementation thereof. More specifically, the present disclosure relates to a system having at least one user application for use during the turnover of operating rooms between patients which provides a streamlined workflow to teams performing the turnover and contextual data to administrators. The user application collects and exports the data tracked to other components of the system for review, storage, analysis, and identification of where improvements may be made in the process. The system additionally allows hospital staff to track the progress of the turnover in real time and allows surgeons to input and store additional information relating to preferences that may be viewed by staff during the turnover process. The system supports user input and workflow action editing of in-progress turnovers from multiple user devices simultaneously, as well as real time tracking of turnovers from a number of additional user devices.

In the present disclosure, the term turnover relates to the cleaning and proper set up of an operating room, generally from when a first patient leaves an operating room to when a second patient enters the same operating room. More specifically, the term turnover refers to any events or tasks that must be completed in an operating room between a first operation and a subsequent operation. More details regarding the application and method may be found below in the description with reference to the corresponding Figures.

The disclosed system 100 aims to promote the coordination and teamwork, as exhibited in Formula 1 pit crews, via teams that prep operating rooms between patients, and thus aims to decrease the time and cost of operating room turnovers for hospitals that use the system.

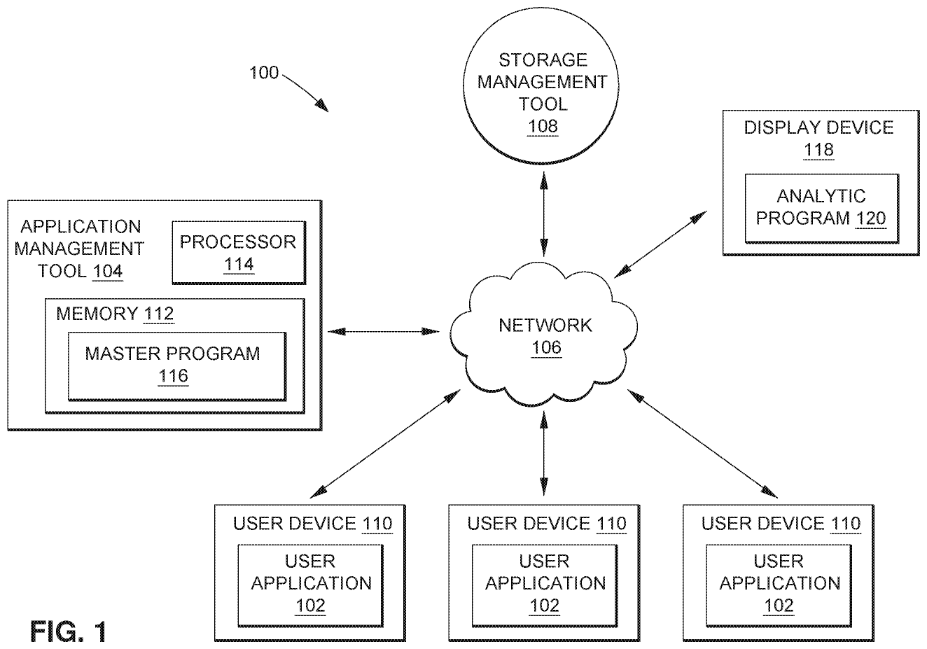

Turning now to FIG. 1, one embodiment of the present disclosure includes a turnover management system 100 incorporating at least an end user turnover management application 102. The system 100 generally includes one or more computing devices, acting as an application management tool 104, a communication network 106, one or storage management tools 108, and one or more end user devices 110. The system 100 is configured to collect categorized inputs from user devices 110, transmit those inputs via the network 106 to the application management tool 104, wherein the inputs are compiled as specified data, and transmitted via the network 106 to the storage management tool 108 for storage. The specified data may be analyzed by the application management system 100 as live data or historical data for a variety of data tracking needs.

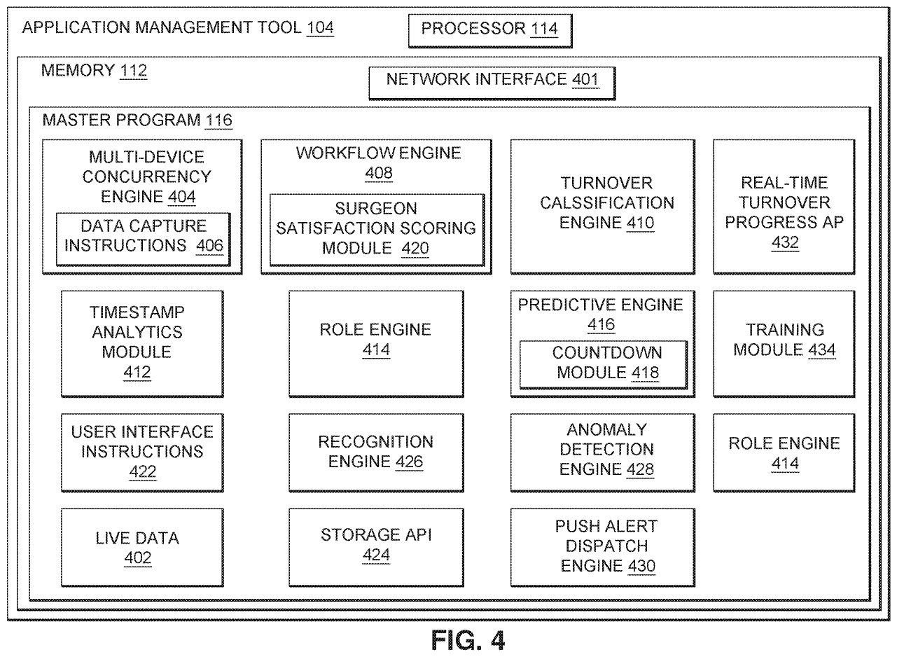

The application management tool 104 may be configured to provide data management functions, scripting functions, data analysis functions, user interface (UI) functions, etc., to the turnover management system 100. As a non-limiting example, the application management tool 104 may be a unitary computing device, which may comprise at least a non-transitory computer readable memory 112 and one or more processors 114. The memory 112 houses the master program 116 generally configured to manage and direct the user applications 102. The master program 116 may have a variety of engines, modules, instructions, APIs, and live data and may configure the processor 114 to execute instructions from the memory 112. Alternatively, the application management tool 104 can be integrated into one or more computing devices by way of a software or a suite of software packages that may configure or program the computing device to operate as application management tool 104. The application management tool 104 may be configured to execute, via one or more computing devices, one or multiple different types of software instructions stored in one or more non-transitory computer readable memories 112 in connection with receiving, processing, and distributing turnover data or relevant visual representations of said data to or from at least one or more user devices 110 and/or the storage management tool 108. The components of the application management tool 104 will be discussed in more detail below in reference to FIG. 4.

The communication network 106 facilitates communications between the user devices 110 and the application management tool 104, as well as between the application management tool and the storage management tool 108. Though the communication network 106 may comprise any wired or wireless network known in the art capable of communication with the application management tool 104, the storage management tool 108, and the one or more user devices 110, illustrate one non-limiting example of a communication network may be wireless internet connection.

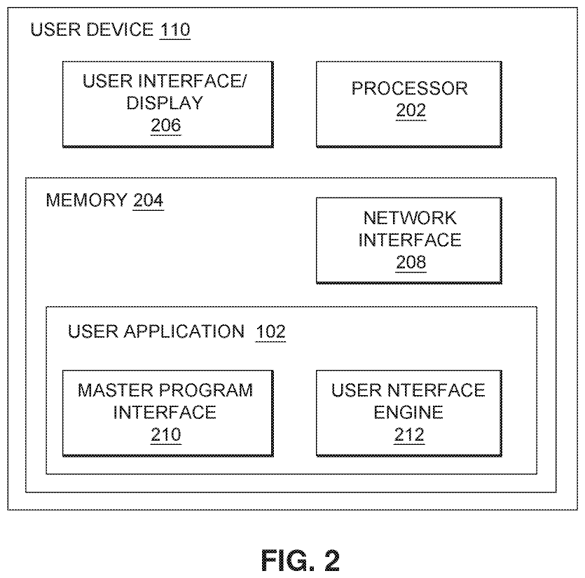

Turning now to FIGS. 1 and 2, the user devices 110 may be configured to display the turnover management user application 102 as executed by the application management tool 104 and transmit user input via the network 106 to the application management tool 104. The user devices 110 may correspond to any type of computing resource that includes a processor 202, computer memory 204, and a user interface or display 206 collectively operating as an endpoint device. The user devices 110 may include any computing resource capable of hosting and running the turnover management user application 102 and may have a network interface 208 configured to allow the user device 110 to be in communication with the network 106, and thus, in communication with the application management tool 104. The user devices 110 may also have a master program interface 210 configured to relay any instructions given by the turnover management tool 104, such that these instructions may be executed by the processor 202. According to one non-limiting example, the user device 110 may be a mobile device. More specifically, this exemplary user device 110 may be a smartphone having a touchscreen display and a camera. The user devices 110 may also include a user interface engine 212 within the memory 204 configured to govern user input and real-time navigation of the user application 102. The interface engine 212 may be subject to various instructions provided by the turnover management tool 104. Alternatively, in other embodiments, the user interface engine 212 may instead be housed in the memory 112 of the application management tool 104 or may be supplementary to user interface engine instructions 422 of the application management tool 104.

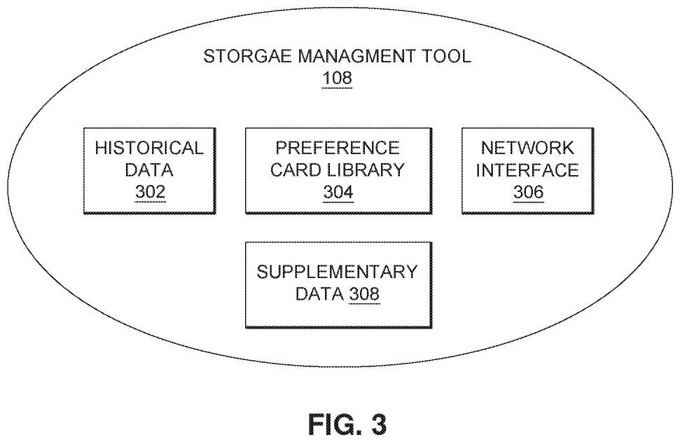

Turning now to FIGS. 1 and 3, the storage management tool 108 may be configured to receive, store, and reformat data required for operation of the turnover management system 100. More particularly, the storage management tool 108 may store historical turnover data 302, and in some embodiments, may also store preference cards 1202 provided by surgeons in a preference card library 304. An example of a preference card 1202 may be seen in FIG. 12. The preference card library 304 will be discussed in further detail below. In certain embodiments, the storage management tool 108 may also be a computing device or may be housed on a computing device having a network interface 306, allowing the storage management tool 108 to receive data provided by the application management tool 104 or another component of the system 100 via the network 106. In other embodiments, the storage management tool 108 may not be or be housed on a computing device. The storage management tool 108 may take any form known in the art so long as data may be stored and so long as the storage management tool 108 may be operatively coupled to the network 106. The storage management tool 108 may generally comprise any storage device, system of storage devices, individual servers, server systems, cloud-based servers, or any other storage medium configured to at least retain large quantities of data or other files and recall specific data using the other components of the turnover management system 100. The storage management tool 108 may also have a cloud redundancy and auto-failover architecture to ensure secure data storage. The storage management tool 108 may also, via the network 106, be in communication with one or more display devices 118, configured to at least display the data housed in the storage management tool 108. The display device 118 may be any device having at least a display, including user devices 110, though in some embodiments the display device may be a separate computing device. The display device 118 may also be configured to allow for additional data input beyond the historical turnover data 302 automatically stored. As a non-limiting example, supplementary data 308 such as financial/cost information may be input using a display device 118 and stored in the storage management tool 108 for the general purpose of providing supplementary contextual data 308 for additional analysis of historical data 302 of past turnovers. Though storage is the main purpose of the storage management tool 108, the storage management tool 108 and/or the display device 118 may also be configured to reformat turnover data, for example into the form of a spreadsheet, and may also be configured to perform sorting or recall functions of data 302, 308 based on given criteria. As a non-limiting example, the storage management tool 108 may be able to display, on a display device 118, data 302, 308 from a specific date range and/or anomalous data based on user input specifying criteria of interest from a display device 110. Another non-limiting example may include creating an exportable log based on data from a specific turnover or based upon data from multiple turnovers.

The display device 118 may also include an analytics program 120 having a historical turnover trend analytics engine, executable on the processor of the computing device. The historical turnover trend analytics engine of the analytics program 120 may access the historical data 302 and the supplementary data 308 from the storage management tool 108 and analyze performance patterns over time, compare turnover classifications across periods, track delay frequencies by room or procedure type, measure improvement trends, identify areas for efficiency improvement, identify bottlenecks, etc. Additionally, the analytics program 120 may be configured to estimate cost savings over time by comparing the historical turnover data 302 against provided supplementary data 308. As a non-limiting example, the historical data 302 may be compared against facility-specific targets and multiplying time reductions by the facility's operating room cost per minute. Alternatively, the analytics program 120 may be hosted by the storage management tool 108, if the storage management tool is configured to act as a computing device having at least a memory and a processor capable of executing instructions from the analytics program 120.

Turning now to FIGS. 1 and 4, FIG. 4 depicts a block diagram of the turnover management tool 104. As mentioned above, the turnover management tool 104 generally comprises at least a processor 114, a memory 112, and the master program 116. The memory 112 may house the master program 116, comprising a variety of engines, modules, instructions, APIs, etc., and a network interface 401. The master program 116 may generally govern and manage operations of the user application 102 and the general flow of information through the system 100. The network interface 401 allows the application management tool 104 to communicate with the various components of the system via the network 106.

Once application management tool 104 receives turnover data from one or more user devices 110 during an in progress turnover, the data may be categorized as live data 402 for the general purpose of real-time analysis. After the turnover is completed, the live data 402 may be transmitted to the storage management tool 108 and recategorized as historical data 302 for retention and historical analysis. It should be appreciated that turnover data need not be so limited as being defined as only either live data 402 or historical data 302, nor does live data 402 only encompass data from an in progress turnover for the purposes of the present disclosure. As a non-limiting example, live data 402 may be defined to include turnover data from an entire day of turnovers instead of data from a single turnover. As such, turnover data may be considered both live data 402 and historical data 302 if the turnover has been completed and it is within the same day.

It should be appreciated that the engines, modules, instructions, and APIs stored in the memory of the various computing devices discussed herein may generate specified instructions executable by at least one processor and, when executed, enable the computing device to perform or otherwise execute the various processes and methods described herein. While particular engines, modules, instructions, and APIs are described herein as being separate from one another, it should be appreciated that various engines, modules, instructions, and API may be combined and/or may have some overlapping functionality. Further, the engines, modules, instructions, and APIs may generate specified instructions capable of being executed by a processor, either individually or in combination, and may be executed in any order so long as the intended outcome may be achieved. It should further be appreciated that the following engines, modules, instructions, and APIs may be specifically categorized as an engine, a module, instructions, or an API, but they need not necessarily be so specifically defined to fit within the spirit and scope of the present disclosure. For example, though data capture instructions 406, which are stored in the memory 112 of the application management tool 104, are described below, it may be better categorized as a data capture engine 406 or a data capture module 406, especially, but not exclusively, for alternative embodiments or variations of the present turnover management system 100. More generally, the labels of engine, module, instructions, and/or API need not be specifically limiting or all-encompassing of the functions of such elements. It should also be appreciated that the order in which the engines, modules, instructions, and APIs are presented below and are described to function sequentially are similarly not meant to be limiting. The engines, modules, instructions, and APIs described below may generate specific instructions to execute processes sequentially, though not necessarily in the exact sequences described below, and/or may generate specific instructions to execute processes simultaneously, constantly, or in loops.

Further, though the turnover management system 100 described herein generally focuses on a system 100 providing a software service for one hospital comprising a multitude of operating rooms, simultaneous turnovers, and a multitude of user devices 110 submitting inputs, this disclosure further contemplates additional, duplicate, and/or alternative elements necessary to support scaling the system to encompass multiple hospitals. The turnover management system 100 described herein may be configured to or may be modified in order to be configured to provide the software service for a multitude of hospitals, each comprising a multitude of operating rooms, simultaneous turnovers, a multitude of user devices 110 submitting inputs, and sensitive patient information. As a non-limiting example, each hospital may have a separate computing device having a software, allowing the computing device to act as a software management tool 104 and further, the storage management tool 108 may be configured to sandbox data from each hospital separately to comply with privacy and healthcare data disclosure regulations.

The multi-device concurrency engine 404 may be configured to control and sort the inputs received by user devices 110 to allow the system 100 to accommodate simultaneous data entry from multiple user devices 110. The multi-device concurrency engine 404 may include the data capture instructions 406 which, when executed by the processor 114, may enable the application management tool 104 to receive inputs from user devices 110 and assign a multitude of classification tags or attributes to each input, allowing for the initial categorization of each input. The data capture instructions 406 may assign tags alone or based upon inputs from various other engines, modules, instructions, and/or APIs. Once an input has been received by the application management tool 104, the workflow engine 408 may be configured to assign tags including, but not limited to, a status tag (i.e., live data 402 or historical data 302), a location tag (i.e., operating room/hospital from which the input originated from), a turnover tag (i.e., turnover number/turnover type), a temporal tag (i.e., time and date), a timestamp tag (i.e., indicating when input was received relative to timer start), an action tag (i.e., action taken/completed), and/or a role tag (i.e., role assignments). The data capture instructions 406 are configured to effectively convert the inputs into data that may be recognized and read by the other data-driven engines, modules, instructions, and APIs in the system. Likewise, the data capture instructions 406 may assign corresponding tags and additional tags to the turnover start and/or completion inputs. These additional tags may include team tags (i.e., identifying the role assignments of all participants), a surgeon tag (i.e., which surgeon the turnover is being completed for), a delay tag (i.e., indication of whether a delay occurred), etc.

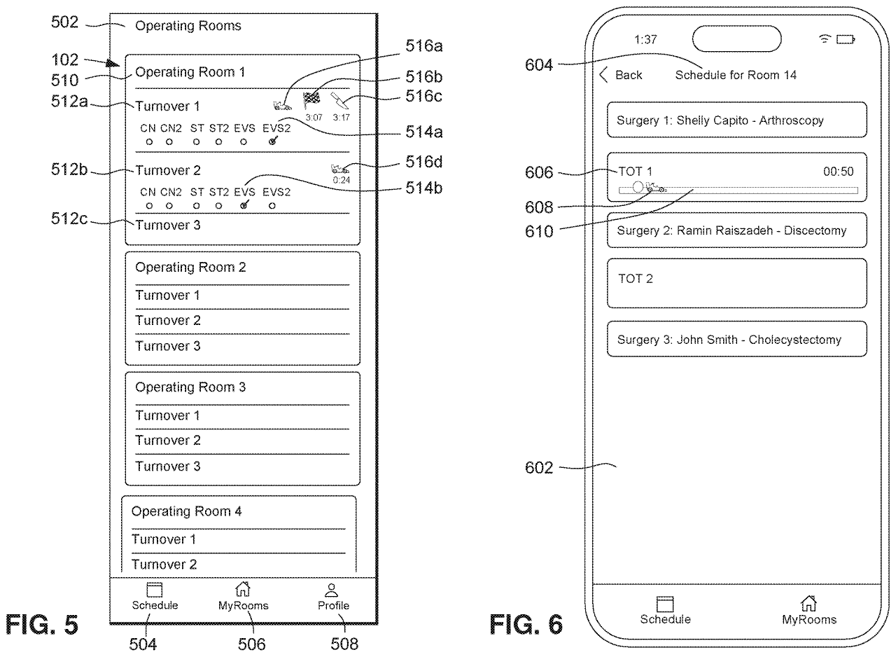

The turnover-classification engine 410, when the generated instructions are executed by the processor, may enable the application management tool 104 to assign classifying performance tags to a completed turnover based upon the total duration of the turnover, then display pictorial representations of each tag on user devices 110. The turnover classification engine 410 may be responsive to inputs from the data capture instructions 406 and may in turn provide corresponding directives for the UI instructions 422 for display. These classifying tags may include time-dependent tiers to classify each completed turnover. As a non-limiting example, turnovers completed in 25 minutes or less may be assigned a first tag represented by a racecar, turnovers completed between 25 minutes and 30 minutes may be given a second tag represented by a turtle, turnovers completed between 30 minutes and 35 minutes may be assigned a third tag represented by a snail, and turnovers completed in over 35 minutes may be assigned a fourth tag represented by a sloth. FIGS. 5, 6, and 7, discussed in more detail below, one example of these pictorial tags, or indicators 516a, 516d, 608, 706. A turnover indicated in the user application 102 as completed will trigger the turnover-classification engine 410 to identify the input and corresponding timestamp representing the total turnover completion time, as indicated by the data capture instructions 406, then automatically tag the turnover based upon the value of the identified timestamp. The UI instructions 422 may then be responsive to the directive from the turnover-classification engine 410 to display the corresponding pictorial representation.

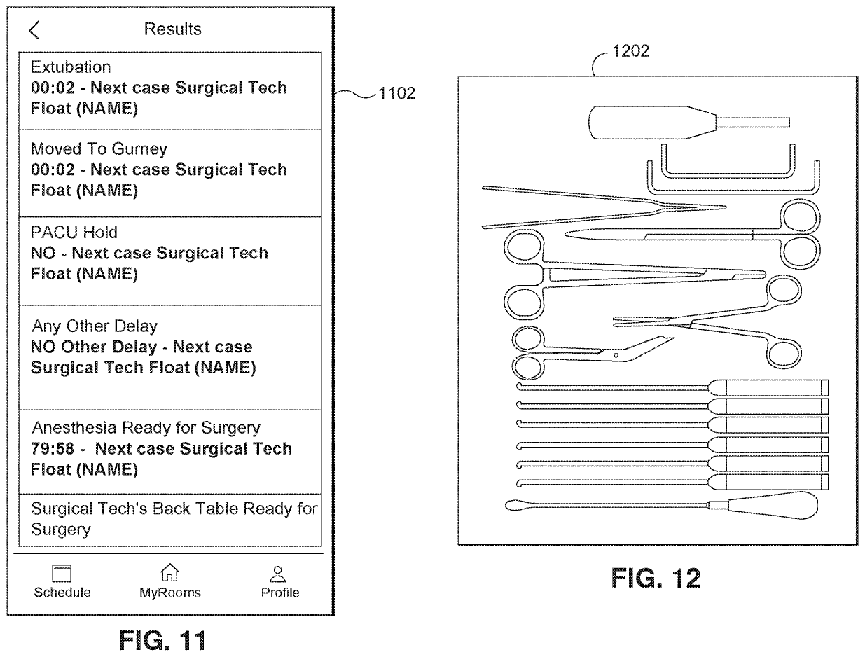

The timestamp analytics module 412, when the generated instructions are executed by the processor 114, may enable the application management tool 104 to analyze inputs from the user devices 110 based upon the timestamp for each workflow action and total turnover duration. Each turnover may have a multitude of timestamps captured relating to each workflow action relative to the start of the main timer. The timestamp analytics module 412 analyzes the inputs based upon the relevant tags assigned by the data capture instructions 406. This allows the timestamp analytics module 412 to determine the length of each workflow state, including each specific workflow action and the turnover as a whole, which may then be analyzed against historical data 302 to determine the efficiency of each workflow state. As a non-limiting example, the timestamp analytics module 412 may output one comprehensive page per turnover containing all event timestamps, associated users and roles, performance classification, documented delays, and next-procedure details. One such example may be seen in the exemplary screenshot 1102 shown in FIG. 11. The timestamp analytics module 412 may further be responsive to inputs from the role engine 416 to analyze live data 402 of one or more specific role assignments with the historical data 302 of one or more of the same or different role assignments. Further, the turnover classification engine 410 may be responsive to inputs from the timestamp analytics module 412, and vice versa, for the analysis of timestamps relating to turnover completion time.

The turnover management tool 104 may have a role engine 414 that, when generated instructions are executed by the processor 114, allows for the capture of a detailed role assignment for each user device 110, and thus user-submitting inputs through the user application 102. This detailed role assignment allows the data capture instructions 406 to attribute data 302, 402 captured for each event to the specific user and selected surgical role. The role engine 414 may include roles such as surgeon, anesthesiologist, circulator, scrub tech, admin, etc. The role engine 414 may attribute the role assignment to each user using one or a variety of identification methods. For example, role attribution may be accomplished by implementing user profiles, requiring a user to login via username and password, biometric identification, passkey authentication, and/or two-factor authentication prior to gaining access to the user application 102. In addition to or in lieu of user profiles, the user application 102 may require the user to input or select identifying information, such as a name or prescribed turnover role, prior to the start of a turnover. An example of a user-input role prompt may be found in the exemplary screenshot 802 shown in FIG. 8. The role engine 414 is configured to create immutable assignments/profiles for each user to uphold accountability, responsibility, and performance attribution through data and audit trails of live data 402 and historical data 301. Once an input signaling completion of a workflow action has been received by the turnover management tool 104, the role engine 414 and the data capture instructions 406 work in conjunction to assign the input with a tag indicating the user and/or role assignment responsible for submitting the input. The role engine 414 may also provide directives to the UI instructions 422, allowing certain role assignments such as hospital administrators to access and view data not accessible by other role assignments.

The turnover management tool 104 may also include a predictive machine learning model and/or predictive engine 416 configured to predict the duration and/or start time of an upcoming turnover and/or the time until completion of an in-progress turnover by performing predictive analytics using historical data 302. The predictive analytics engine 416 may consider a multitude of factors including the indicators assigned to inputs and completed turnovers by the data capture instructions 406, as well as other external or deduced factors such as the number of other turnovers being performed prior to or in conjunction with the turnover of interest, the completion time of the prior turnover in the same operating room, delay patterns, etc. The predictive engine 416 may establish an analytical foundation for calculating average turnover durations by procedure type and/or assigned surgeon, by identifying recurring bottlenecks, and by forecasting completion times for active turnovers. The calculations for average turnover durations may also inform the parameters of the turnover classification engine 410, allowing turnovers to be scored based upon dynamic historical data 302. Further, forecasting completion times for active turnovers may allow the turnover management tool 104 to create future predictive schedules and may notify staff when dynamic schedule changes may increase efficiency, allowing for healthcare staff to be more informed about delays when they occur. The predictive engine 416 may also monitor real-time workflow progress across all operating rooms and, in conjunction with the UI instructions 422, send notifications to staff to suggest personnel allocation to critical-path tasks based on completion states, team availability, and predicted bottlenecks. The predictive engine 416 may also be configured to train and re-train itself continuously based upon new historical data 302 it has not yet ingested.

The predictive engine 416 may also include a countdown module 418 that, when the generated instructions are executed by the processor 114, displays a countdown timer on the user application, indicating when an in-progress turnover is predicted to be completed. The countdown module 418 may receive a turnover completion prediction from the predictive engine 416 and may then direct the UI instructions 422 to display a standard running countdown on user devices 110 until the countdown is directed to be updated. This process may be constantly updating with the predictive engine 416 updating the prediction when certain conditions have been met, such as a specified time period between updates or updating when workflow actions are input as completed. An updated prediction may cause the countdown module 418 to direct the UI instructions 422 to update the countdown on the user application 102 given certain conditions, such as if the prediction is a certain time value different from the current running countdown. The countdown module 418 may also be configured to track and direct display of multiple timers/countdowns at once. As a non-limiting example, the countdown module 418 may direct the UI instructions 422 to display a predictive countdown until turnover completion simultaneously with a predictive countdown until the operating room is ready for another team such as the surgical tech team or the EVS team. As such, the countdown module 418 may also be configured to direct the UI instructions 422 to assign a text label to the countdown timers to eliminate confusion regarding which event the countdown is indicating.

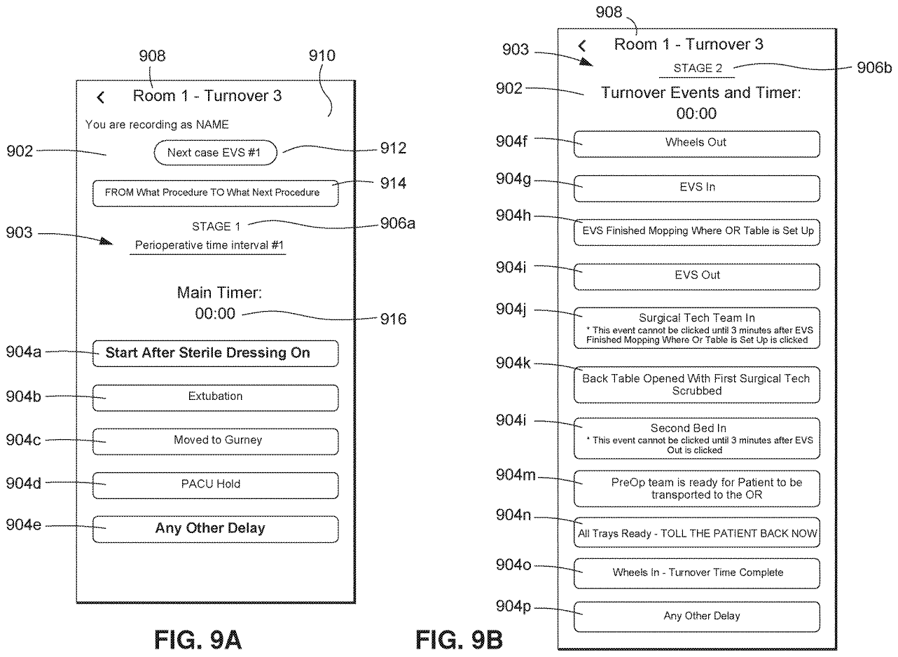

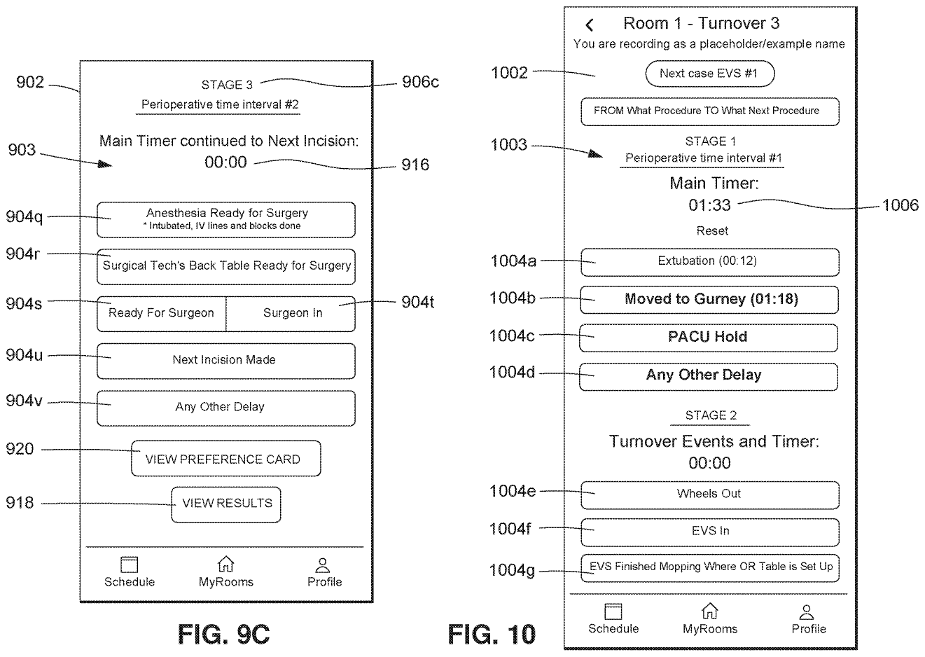

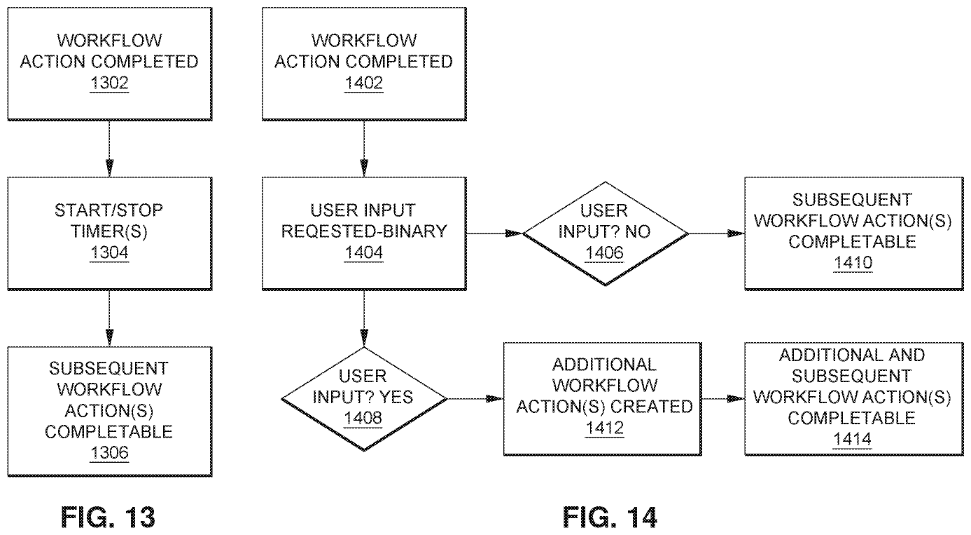

The workflow engine 408, when the generated instructions are executed by the processor 114, may allow the application management tool 104 to track and dynamically manage the progress of multiple completed turnovers, in-progress turnovers, and/or upcoming turnovers by identifying and executing certain events when specific criteria are met. The workflow engine 408 may be state-driven in nature, with the events executed and instructions generated being dependent upon the progress or state of the workflow as a whole, the workflow stage(s), and/or the workflow action(s). One implementation of the user interface for this state driven workflow 903, 1003 may be found in FIGS. 9A-10. Based upon information from the data capture instructions 406 indicating these criteria, the workflow engine 408 may provide directives to other engines, modules, instructions, and APIs to execute these events. As a non-limiting example, the workflow engine 408 may receive input from the data capture instructions 406 that a specific workflow action has been completed, the workflow engine 408 may then recognize that the workflow action completed requires a secondary input, a popup, creation of additional workflow action, a predetermined time-based hold before the next action may be completed, etc. These workflow actions requiring another criterion for completion or progression beyond a single user input may also be referred to as non-standard workflows or non-standard workflow actions. The workflow engine 408 will then provide directive to the UI instructions 422 to prompt the user with the secondary input or popup. As non-limiting examples, a popup or secondary input may prompt the user to input a binary choice, input freeform text, input a selection from a list, etc. An example of a popup may be seen in the screenshot 802 shown in FIG. 8. Further, flowcharts depicting select examples of these processes may be seen in FIGS. 13 and 14, which will be discussed further below. As another non-limiting example, the workflow engine 408 may receive input from the data capture instructions 406 that a specific workflow action has been completed, the workflow engine 408 may then recognize that the workflow action completed indicates a crucial step requiring an additional visual indication to be displayed for all hospital staff. These additional visual indicators may, for example, be shown on the schedule, shown in FIG. 5, to indicate that one stage is about to end and another is about to begin. The workflow engine 408 may then provide directives to the UI instructions 422 to display a corresponding visual indication and/or text label. Further, the workflow engine 408 may be subject to directives from the role engine 414 and may provide directives to the UI instructions 422 to generate and display a workflow action checklist specific to each assigned role in the turnover process.

The workflow engine 408 may also have a surgeon satisfaction scoring module 420 which, when the generated instructions are executed by the processor 114, prompts the surgeon assigned to a turnover to score his or her satisfaction with the turnover based upon a multitude of factors. Some predetermined time after the turnover of interest has been completed, the surgeon satisfaction scoring module 420 may direct the UI instructions 422 or the push alert dispatch engine 430 to prompt the surgeon to score the turnover of interest. If initiated by surgeon input, the surgeon satisfaction scoring module 420 may display a variety of factors upon which the turnover may be scored numerically, as well as freeform text boxes for the surgeon to elaborate upon the numerical scoring. As a non-limiting example, the surgeon satisfaction scoring module 420 may prompt the surgeon to rate the quality of the speed, the cleanliness, the instrument preparation, etc. of the turnover and/or the quality of the work of each turnover team, as well as the quality of the turnover as a whole. A freeform text box may additionally prompt the surgeon to leave any additional comments. Once submitted, the surgeon satisfaction survey may be stored in the storage management tool 108 with the rest of the corresponding data 302, 402 associated with the turnover.

The UI instructions 422, when executed by the processor 114, allows the application management tool 104 to display and make dynamic changes to the user interface of the user application 102. The UI instructions 422 may be configured to instruct the user device 110 to display or not display real-time/live data 402, real-time turnover progress indicators, admin dashboards, reports, workflow prompts, etc. based upon a number of predetermined factors. Generally, the UI instructions 422 are responsive to inputs from other engines, modules, instructions, and APIs and comprise the last step before display on user devices 110. As a non-limiting example, based upon directives from the role engine 414, the UI instructions 422 may or may not display information or workflow actions on a specific user device 110 based upon the user's/user device's 110 role assignment. As another non-limiting example, the UI instructions 422 are responsive to directives from the turnover classification engine 410, indicating which pictorial representation or indicator 516a, 706 should be displayed for completed turnovers. As is described above, the UI instructions may also be responsive to directives from the workflow engine 408, allowing for a dynamic UI based upon the completion of certain workflow actions and/or the fulfillment of specific criteria.

The storage API 424, when the generated instructions are executed by the processor 114, allows the application management tool 104 to export data to or from the storage management tool 108 or another external system. Once a turnover is completed, the storage API 424 then automatically removes all patient identifying information from the turnover data and transmits that data via, for example, HTTP POST or RESTful HTTP communication to the storage management tool 108 or an external system. These external systems may include, but are not limited to EHR (electronic health record) systems, hospital scheduling systems, etc. In addition to raw data being transmitted, the storage API 424 may also transmit any reports, audit logs, or other similar documents requested by users or automatically created by, for example, the timestamp analytics module 412 to the storage management tool 108 for storage. In other words, any information, files, data, logs, etc. created by or passed through the application management tool 104 may be transmitted to the storage management tool 108 or another external system via the storage API 424. Likewise, the storage API 424 may also be configured to transmit data, files, logs, etc. or copies of such from the storage management tool 108 to the application management tool 104 or an external system for analysis. Once analysis is completed the information and resulting analysis report, log, or other document is transmitted back to the storage management tool 108.

The recognition engine and/or recognition machine learning model 426, when the generated instructions are executed by the processor 114, may allow the application management tool 104 to recognize and identify whether certain conditions have been met based upon a provided input. The provided input may include RFID or BLE tags present on certain instruments to track movement of the instrument and the location of the instrument. The provided input may also include a photograph captured by a user device 110. The recognition engine 426 may utilize the movement tracking of the RFID/BLE tags or visual recognition of a photograph to determine whether conditions have been met to approve or deny the user completion of an action. As a non-limiting example, the recognition engine 426 may be configured to track the location of the RFID or BLE tags associated with the instruments indicated on the relevant surgeon preference card. The recognition engine 426 may indicate that the condition required to complete the instrument prep workflow action is satisfied when the RFID or BLE tags indicate that the locations of all necessary instruments are within the correct operating room. As another non-limiting example, upon completion of a cleaning task, the workflow engine 408 may prompt the user to take a photo of the area cleaned. The recognition engine 426 may then review the photo and identify whether the cleaning was satisfactory or unsatisfactory.

As a yet further non-limiting example described in detail, the recognition engine 426 may recognize whether instruments present in a photograph match those of a surgeon's preference card or exemplary photo from the preference card library 304. This may be achieved utilizing a multitude of engines, modules, instructions, and APIs. First, when the turnover profile is created prior to start, the data capture instructions 406 may tag the turnover as being assigned to a first surgeon and for a first procedure. This information is then received by the workflow engine 408 and UI instructions 422 to create a workflow UI based upon the information provided. The workflow engine 408 may identify that the surgeon preference card was not yet provided and may then request the storage API 424 to search the preference card library 304 stored in the storage management tool 108 for the preference card corresponding to the first surgeon and the first procedure. If the storage API 424 identifies a preference card and corresponding photo meeting these criteria, then it is sent to the workflow engine 408 and UI instructions 422 to be implemented in the workflow UI by implementing an accessible preference card and photo and, optionally, by updating the workflow actions to create actions relating to each instrument requested. Users may then view the preference card and photo prior to turnover start. During the turnover, once the specified workflow action has been completed, the input will be captured, tagged by the data capture instructions 406, and transmitted to the workflow engine 408. The workflow engine 408 then recognizes that a preference card and a photo are present, and it directs the UI instructions 422 to prompt the user to take a photo of the instruments. Once submitted, the photo is transmitted to the recognition engine 426, wherein the user photo input is compared to the photo from the storage management tool 108. The recognition engine 426 identifies individual instruments through visual pattern recognition and flags missing items, incorrect placements, or substitute instruments. The recognition engine 426 may then direct the push alert engine 430 and/or UI instructions 422 to indicate that the instruments presented in the photograph are unsatisfactory and may direct the data capture instructions 406 to refuse a completion input of the workflow action until the photograph is deemed satisfactory. The recognition process may repeat until the photograph is deemed satisfactory, then the recognition engine 426 may direct the data capture instructions 406 and/or the workflow engine 408 to allow the successful completion of the workflow action.

The anomaly detection engine 428, when the generated instructions are executed by the processor 114, allows the application management tool 104 to capture, categorize, and store freeform inputs from user devices 110 which may, for example, explain any delays that may have occurred based upon a variety of input factors. The anomaly detection engine 428 may work in collaboration with the workflow engine 408 and the UI instructions 422 to display a textbox prompt to the user that previously indicated that, for example, a delay occurred. As a non-limiting example, after certain workflow actions, the workflow engine 408 will direct the UI instructions 422 to display a secondary input or popup prompting the user to select from a binary “Yes” or “No” regarding whether a delay has occurred. If the user indicates a “Yes” input, the data capture instructions 406 give the input the proper tag and the anomaly detection engine 428 directs the UI instructions 422 to display a textbox prompt for the user to explain the delay. The freeform response is recorded along with tags assigned by the data capture instructions 406 and/or the anomaly detection engine 428, indicating corresponding identifiers such as the responsible role, timestamp, frequency, duration, and turnover context, creating labeled records that classify delays by type, such as EVS-related, equipment-related, staffing-related, etc. After the delay explanation input has been received, the anomaly detection engine 428 may compare the assigned tags and freeform response against historical data 302, more specifically historical delay explanations, in an effort to identify patterns or keywords. Based on this analysis, the anomaly detection engine 428 then assigns the turnover a delay tag, and the delay explanation and corresponding tags are stored in the storage management tool 108 with the corresponding turnover data.

The push alert dispatch engine 430, when the generated instructions are executed by the processor 114, allows the application management tool 104 to send push alerts to user devices 110 when certain criteria are met. The push alert dispatch engine 430 relies upon directives or inputs from other engines, modules, instructions, and APIs to initiate execution of push alerts. As a non-limiting example, the push alert dispatch engine 430, in conjunction with the role engine 414 and workflow engine 408, may be configured to send push alerts to certain role assignments regarding the status of completed, in-progress, or upcoming turnovers. More specifically, the push alert dispatch engine 430 may receive a directive from the workflow engine 408 and/or predictive engine 416 indicating that a specific workflow action is taking longer than anticipated or that a specific workflow action may have been inadvertently skipped. The push alert dispatch engine 430 may then request input from the role engine 414 to determine the role assignments currently performing the turnover and push an alert to the corresponding user devices 110, prompting the users to input the completion of the workflow action, if the workflow action is indeed completed. As another non-limiting example, the push alert dispatch engine 430 may be responsive to inputs from the predictive engine 416 indicating that efficiency may be increased with dynamic personnel reallocation. The push alert dispatch engine 430 may then request input from the role engine 414 to determine the appropriate role assignments to notify and push an alert to the corresponding user devices 110.

The application management tool may also include a real-time turnover progress API 432, which may be configured to transmit real-time, live turnover data 402 to external programs or systems such as hospital dashboards. The real-time turnover progress API 432 may utilize some or all of the turnover management system 100 infrastructure to transmit in-progress/live turnover data 402, information, and/or analysis to an external program or system. For instance, the real-time turnover progress API 430 may transmit information from the various engines, modules, instructions, and APIs of the turnover management system, such as turnover status data (i.e., not started, in-progress, or completed), workflow progress data (i.e., workflow actions completed, delay occurrence), estimated turnover completion time, projected turnover start time, assigned turnover data (i.e., room number, roles assigned, procedure type, surgeon assigned), etc., based upon requests from the external program. This may allow other hospital staff such as reception staff and other non-turnover staff to view the status of multiple turnovers without requiring access to the user application 102.

The turnover management user application may also include a dedicated training module 434 integrated into the user application. The training module 434 may use the general framework of the step-by-step turnover workflow and the various other engines, modules, instructions, etc. of the system 100 to guide new operating room staff through the real-time turnover workflow actions, without factoring the training data into the tracking, storage, and analytics of the turnover events described above. When activated, the training module 434, in conjunction with other engines, modules, and instructions may present each workflow action with contextual explanations and instructional overlays, describing what actions to perform, why each action matters for overall timing, and common mistakes to avoid. The training module 434 validates action timing and sequencing, providing immediate feedback when trainees skip required actions or confirms actions out of order, and tracks trainee progress through completion metrics showing which workflow stages, states, or actions require additional practice.

Turning now to FIGS. 5-11, exemplary implementation of the turnover management system 100 may be seen in a variety of screenshots of exemplary embodiments of the user application 102. These Figures generally show how the user application 102 may appear while in use.

As can be seen in FIG. 5, an exemplary screenshot 502 of the user application 102 taken from a user device 110 may be seen. The user application 102 may generally have three main tabs: ‘Schedule’ 504, ‘MyRooms’ 506, and ‘Profile’ 508. FIG. 5 depicts an exemplary screenshot 502 of the ‘Schedule’ tab 502 wherein the user may see a list of all operating rooms 510 and all corresponding planned turnovers 512a, b, c for the current day. More specifically, FIG. 5 depicts how the ‘Schedules’tab 504 may display a preview of the status of a completed turnover 512a, an in-progress turnover 512b, and a future turnover 512c.

As can be seen in FIG. 5, role status information 514a, b and one or more indicators 516a, b, c, d may appear with a corresponding turnover 512a, b, c to give a preview of important information relating to each turnover 512a, b, c. The role status information 514a, b displayed may indicate how many users have indicated that they will be participating in the specific turnover 512a, b, c and what roles they have indicated they will fill. For instance, in the present screenshot 502, the role status information 514a of turnover 512a indicates that the role of EVS #2 has been filled and the role status information 514b of turnover 512b indicates that the role of EVS #1 has been filled. The user interface instructions 422 may be subject to directives from the role engine 414 and the data capture instructions 406 to provide accurate role status information 514a, b.

The indicators 516a, b, c, d may provide high-level information regarding in-progress 512a and completed turnovers 512b. These indicators 516a, b, c, d may include simple shapes, pictograms, colors, numbers, words, or a combination thereof. The user interface instructions 422 may be subject to directives from at least the turnover classification engine 410 to display these indicators 516a-c. In one embodiment shown in screenshot 502 of FIG. 5, indicators 516a, b, and c depict a car, a flag having a timestamp, and a knife having a timestamp. Indicator 516b conveys to the user that turnover 512a is complete and how much time was taken to complete the turnover 512a. Indicator 516a conveys to the user that the time taken to complete the turnover 516a is considered “fast”, according to a predetermined threshold. Indicator 516c conveys to the user that the next incision was made and how much time elapsed between turnover completion and incision. Indicator 516d depicts a car with a timestamp, conveying to the user that turnover 512b is in progress and the amount of time that has elapsed since the start of the turnover 512b. A variety of instructions and engines, such as the data capture instructions 406, the workflow engine 408, timestamp analytics module 412, turnover classification engine 410, and user interface instructions 422 may work in conjunction to display these indicators 516a, b, c, d.

In general, any hospital staff using the user application 102 may view whether a turnover is not started, in progress, or completed. In certain embodiments, select hospital staff assigned to oversee or perform a specific turnover may view more detailed in-progress data about that specific turnover. For example, hospital staff assigned to oversee a specific turnover may view steps being completed in real time while the hospital staff assigned to perform the same turnover may input data and edit action progression from multiple devices. In other embodiments, as is shown in FIG. 6, depicting a screenshot 602 of user application 102, selecting an operating room 604 wherein a turnover 606 is in progress from the ‘Schedule’ tab 504 may depict an indicator 608 moving along a progress bar 610 from left to right, indicating the real-time progression of the turnover 606 in progress.

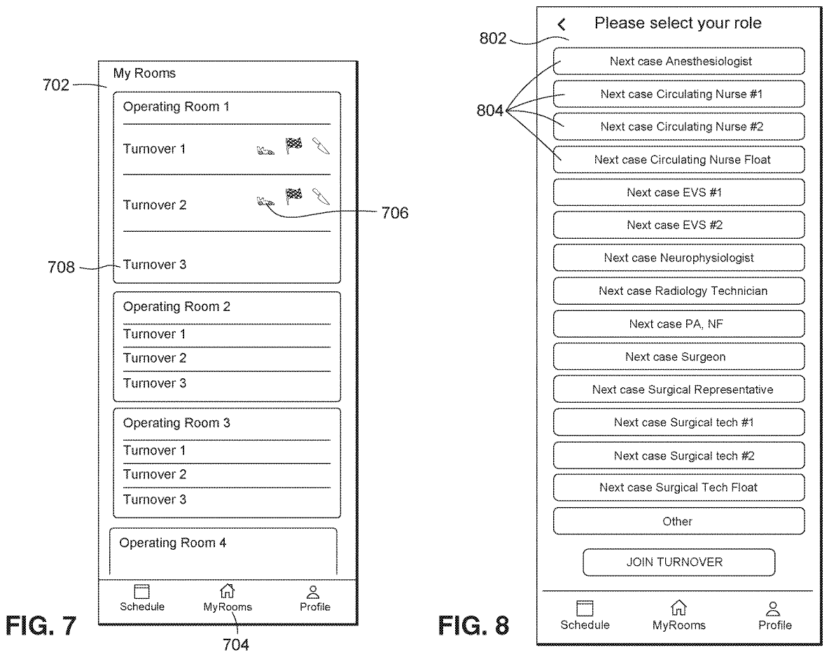

FIG. 7 depicts a screenshot 702 of the ‘MyRooms’ tab 704 of the user application 102. As may be seen, the ‘MyRooms’ tab 704 may allow the user to see which turnovers they are assigned to perform or oversee for the day. The ‘MyRooms’ tab 704 may also depict indicators 706 similar in form, function, and implementation to those displayed on the ‘Schedule’ tab. The turnovers may have multiple staff members assigned to perform the turnover as well as multiple staff assigned to oversee the turnover. The user application 102 supports data input and action editing from multiple users on multiple user devices 110 simultaneously, as well as real time data tracking of in-progress turnovers by multiple users on multiple devices 110.

From the ‘MyRooms’ tab 704, the user may select the next turnover 708 that they are working or overseeing to start or view a specific turnover. The user may then be prompted to select a role 804, which may be seen in FIG. 8, depicting an exemplary screenshot 802 of the role selection user interface. Once a role 804 input has been recorded on a user device 110, the data capture instructions 406 assigns the input corresponding tags, and the role engine 414 assigns the role 806 to the user's profile for the specified turnover. Once a role 804 has been selected, the user may then be presented with a list of workflow actions that may be completed in a particular order, or a turnover workflow 903.

FIGS. 9A-C depict an exemplary screenshot 902 of the user interface of the user application 102 displaying an example of a turnover workflow 903, prior to turnover start. The turnover workflow 903 may include a plurality of workflow actions 904a-v separated into stages 906a, b, c. In the present embodiment depicted in FIGS. 9A-C, the turnover workflow 903 includes three stages 906a, b, c, each stage 906a-c having a set of workflow actions 904a-e, 904f-p, 904q-v to be completed within each stage 906a-c. Generally, the turnover workflow 903 may be state-driven, depending upon execution of instructions generated by the state-driven workflow engine 406. In certain embodiments, the turnover workflow 903 may be defined by three categories of states or events: the workflow as a whole 903, stages 906a-c, and actions 904a-v. In the present embodiment, the turnover workflow 903 or workflow state contains three stages 906a-c or stage states and each stage 906a-c contain a plurality of actions 904a-v or action states. It is contemplated herein that, state categories need not be sub-states of another state nor do categories of state need to be dependent upon other categories of state to fit within the spirit and scope of the present disclosure. The specific workflow actions 904a-v and descriptions depicted herein are purely exemplary and are not intended to represent all possible workflow actions that may be implemented in the present system 100.

Above the turnover workflow 903, the turnover label 908, user name 910, and user role 912 may be displayed. A textbox 914 or plurality of textboxes may be provided and may allow the user to input freeform text to contextualize the turnover. As is shown in FIG. 9A, the user may input the operations the current turnover is occurring between. Other embodiments of the textbox 914 or additional textboxes may, for example, prompt the user to input the name of the next surgeon, the name of the next anesthesiologist, the names of the individuals in the operating room team, or other freeform data to contextualize the turnover.

The turnover workflow 903 may include at least three types of timers, each corresponding to the states above, to track workflow: a main timer 916 tracking the total time duration from start to finish, one or more stage timers 918 tracking the time duration of each stage, and a plurality of action timers tracking the time duration of each workflow action 904a-v. The action timers may or may not be visible to the user on the user application 102 display.

Each workflow action 904a-v may correspond to a specific real-world action the user may carry out in the operating room and may generally follow one of many action sequences, some of which may be seen in FIGS. 13 and 14 and will be discussed in more detail below. Generally, a user may select or tap a workflow action 904a-v once the corresponding real-world action is completed to provide user input to the system 100 that the action 904a-v is complete. As may be seen, for example, in FIG. 9A, workflow actions 904a and 904e that may be selected or input as complete, or completable actions 904a, e may be visually distinct from those workflow actions 904b, c, d that may not yet be input as complete, or non-completable actions 904b-d. For instance, completable actions 904a, e may be displayed in darker shades than non-completable actions 904b-d. In other embodiments, completable actions 904a, e may have additional visual indications that the action 904a, e may be completed. As a non-limiting example, completable actions 904a, e may flash a color or colors in predetermined intervals such that the user may more easily identify the actions 904a, e as completable. Generally, these non-completable actions 904b-d may require satisfaction of a predetermined criterion or predetermined criteria before these actions 904b-d may become completable. In the present embodiment, select workflow actions 904e may additionally be classified as an optional action. An optional action 904e may not be required to be completed to progress through the workflow and, if necessary, may be completed at any point during the action's 904e respective stage 906a. These optional actions 904e may allow, for instance, users to indicate whether a delay has occurred. If a delay occurs, the user may complete this optional action 904a and may be prompted with a textbox to explain the delay. If a delay has not occurred, the user may choose to bypass this optional action 904e or complete the optional action 904e to formally indicate that a delay has not occurred. In the present embodiment shown in FIG. 9A, these optional actions 904e are not visually distinguishable from other completable actions 904a, e, other embodiments of the present disclosure may include distinct visual indicators.

Turning now to FIG. 10, an exemplary screenshot 1002 of the turnover workflow 1003 of an in-progress turnover is shown. Generally, as workflow actions 1004a-g are completed by a user, the completed actions 1004a, b may also be visually differentiated from non-completed actions 1004c-g to indicate that they are completed. The completed actions 1004a, b may be visually differentiated from non-completed actions 1004c-g by, for example, displaying how long from the start of the ‘Main Timer’ 1006 it took to complete, by changing the color of the action 1004a, b, or by modifying any or any combination of visual attributes. Once actions 1004a, b are completed, any subsequent actions that may be completed next in sequence may then become completable actions 1004c, d.

If a completed action 1004a, b is completed on accident, completion of the action 1004b may be reversible. For instance, a completed action 1004b may be selected or tapped again to undo the completion input and may remain reversable for a predetermined time period, such as 30 seconds, after the initial completion input. Similarly to completable actions 1004c, d and non-completable actions 1004e-g, reversible completed actions 1004b may be visually distinct from non-reversible completed actions 1004a. For instance, reversable completed actions 1004b may be shown in shades darker than that of non-reversable completed actions 1004a.

Turning back to FIGS. 9A-C, once all workflow actions are marked as complete, the data may then be exported to the storage management tool 108. In some embodiments, the data may be imported into a spreadsheet prior to being exported to the storage management tool 108. In further embodiments, the user may be required to select a prompt to import the data into a spreadsheet or export the data to the storage management tool 108. After completion of a turnover, a user may select a ‘View Results’ 918 button wherein an overview of the data may be viewed; FIG. 11 depicts an exemplary screenshot 1102 of this data overview. The data may be presented, for example, in list form, in table form, in the form of a spreadsheet, etc. FIGS. 9A-C generally exemplify one embodiment of the workflow actions 904a-v that may be edited and completed via the user application 102 on multiple user devices 110 simultaneously by all hospital staff assigned to perform a specific turnover.

The proposed system 100 likewise presents a solution to the problem associated with incorrect or missing equipment in the operating room. The system 100 may allow surgeons to upload and store their preference cards 1202, an example of which may be seen in FIG. 12, in the storage management tool 108 ahead of time. The surgeon may upload specific and unique preference cards 1202 to the system 100, designating particular cards 1202 for each scheduled turnover in each operating room on the user application 102 via a user device 110 or via another computing device in communication with the system 100, depending on what type of procedure the operating room will need to be prepared for. Uploading these preference cards 1202 well before the turnover is scheduled may allow the personnel assigned to perform or oversee these turnovers to view the preference cards 1202, locate and prepare the equipment needed, and ask clarifying questions before the turnover begins. As may be seen in FIG. 9C, the user may select ‘View Preference Card’920 to see the preferences of the surgeon during or before the turnover.

The system 100 also has additional features for special or circumstance-dependent actions. Flowchart depictions of a selection of these non-standard workflows or non-standard workflow actions may be seen in FIGS. 13 and 14. The flowcharts depicted in FIGS. 13 and 14 are not intended to depict all non-standard workflows that may be implemented in the present system 100. Instead, FIGS. 13 and 14 depict select examples of possible non-standard workflows that may be representative of a plurality of non-standard workflows that may be implemented using the turnover management system 100 described herein. Further, the flow charts depicted in FIGS. 13 and 14 may be representative of one or multiple non-standard workflow actions.

One such non-standard workflow action, shown in FIG. 13, depicts an example wherein a workflow action may be input as completed (step 1302), which may cause one or more timers to start or stop (step 1304), and allow one or more subsequent workflow actions to become completable (step 1306). For instance, referring back to FIG. 9A, completion of action 904a may cause the main timer 916 and the stage one timer (not shown) to start, and may allow actions 904b and 904c to become completable.

In another example shown in FIG. 14, upon completion of a workflow action (step 1402), the user may be prompted with a binary yes/no input choice (step 1404) by, for example a popup. If the user inputs no (step 1406), the following workflow actions become completable (step 1410). If the user inputs yes (step 1408), an additional workflow action is created (step 1412) and implemented into the workflow, and the additional and/or other subsequent workflow actions become completable (step 1414).

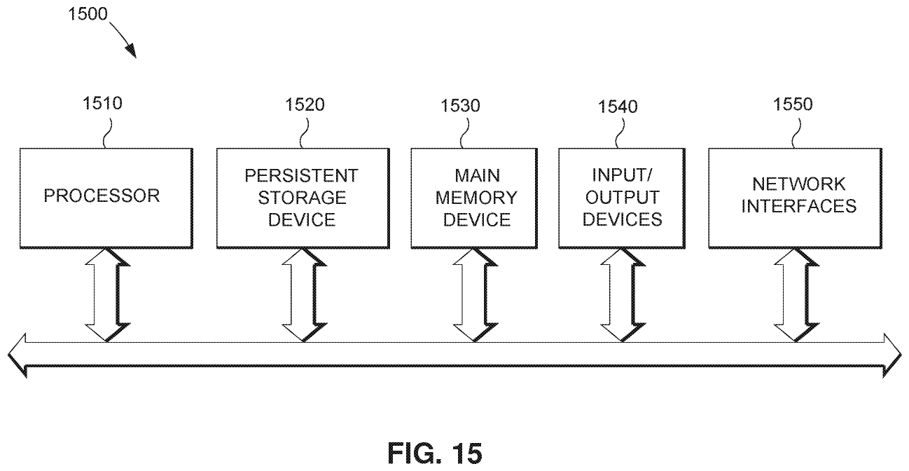

By way of example, FIG. 15 is a high-level block diagram of an exemplary apparatus 1500 that may be used to implement systems and methods described herein. The apparatus 500 may comprise a processor 1510 operatively coupled to a persistent storage device 1520 and a main memory device 1530. The processor 1510 may control the overall operation of apparatus 1500 by executing computer program instructions that define such operations. The computer program instructions may be stored in persistent storage device 1520, or other computer-readable medium, and loaded into main memory device 1530 when execution of the computer program instructions is desired. For example, the application management tool 104, amongst various other components of the system 100 may comprise one or more components of the apparatus 1500. Thus, the operations described throughout the present disclosure can be defined at least in part by the computer program instructions stored in main memory device 1530 and/or persistent storage device 1520 and controlled by processor 1510 executing the computer program instructions. For example, the computer program instructions can be implemented as computer executable code programmed by one skilled in the art to execute an implementation of an algorithm defined by the operations described herein. Accordingly, by executing the computer program instructions, the processor 1510 may execute an algorithm defined by the described operations. The apparatus 1520 may also include one or more network interfaces 1550 for communicating with other devices within the system 100 or outside the system via a network such as the network 106. The apparatus 1500 may also include one or more input/output devices 1540 that enable user interaction with the apparatus 1500 (e.g., display, keyboard, mouse, speakers, buttons, etc.).

Processor 1510 (which may exemplify, in whole or in part, one or more of the processors 114, 202 described herein) may include both general and special purpose microprocessors, and may be the sole processor or one of multiple processors of apparatus 1500. Processor 1510 may comprise one or more central processing units (CPUs), for example. Processor 1510, persistent storage device 1520, and/or main memory device 1530 may include, be supplemented by, or incorporated in, one or more application-specific integrated circuits (ASICs) and/or one or more field programmable gate arrays (FPGAs).

Persistent storage device 1520 and main memory device 1530 may each comprise a tangible non-transitory computer readable storage medium. Persistent storage device 1520, and main memory device 1530, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 1540 may include peripherals, such as a printer, scanner, display screen, etc. For example, input/output devices 1540 may include a display device such as a cathode ray tube (CRT), plasma or liquid crystal display (LCD) monitor for displaying information to a user, a keyboard, and a pointing device such as a mouse or a trackball by which the user can provide input to apparatus 1500.

One skilled in the art will recognize that an implementation of an actual computer or computer system may have other structures and may contain other components as well, and that FIG. 15 is a high-level representation of some of the components of such a computer for illustrative purposes.

The above description is given by way of example, and not limitation. Given the above disclosure, one skilled in the art could devise variations that are within the scope and spirit of the invention disclosed herein, including various ways of developing and implementing an application to improve efficiency and consistency of turnover times in operating rooms and methods thereof. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A method for collecting and displaying time related user input data on multiple mobile devices comprising the steps:
providing at least one processor for communicating with and collecting user input data from one or more mobile devices;
receiving a user input from a user device to initiate a time sequence, wherein the time sequence denotes the time elapsed since a start time input;
displaying the start time input on one or more mobile devices;
receiving a first categorized user input from a mobile device at a first time input;
recording a first timestamp corresponding to the first time input and the first categorized user input;
displaying the first timestamp with the corresponding first categorized user input on one or more mobile devices;
receiving a second categorized user input from a mobile device at a second time input;
recording a second timestamp corresponding to the second time input and the second categorized user input;
displaying the second timestamp with the corresponding second categorized user input on one or more mobile devices;
receiving user input from a mobile device to end the time sequence, creating an end time input, recording an end timestamp corresponding to the end time input, and terminating the time sequence; and
displaying the end timestamp with the corresponding end time input on one or more mobile devices.

2. The method of claim 1, wherein upon receiving the second categorized user input from a mobile device, a countdown timer is started and wherein, upon depletion of the countdown timer, the user is notified via a mobile device.

3. The method of claim 1, wherein upon receiving the second categorized user input, a popup notification is displayed, requiring user input to dismiss.

4. The method of claim 1, wherein upon receiving the second categorized user input, a popup notification is displayed, requiring user input of one of two options to dismiss.

5. A turnover management system for collecting and storing turnover data, the turnover management system comprising:
at least one computer readable non-transitory memory storing software instructions; and
at least one processor, coupled with the at least one memory, that performs operations upon execution of the software instructions, the operations comprising:
receiving a first user input from a first user device via a network;
deriving a plurality of first attributes from the first user input;
converting the first user input to a first instance of data defined by the plurality of first attributes;
compiling the first instance of data into a data set;
receiving a second user input from a second user device via the network;
deriving a plurality of second attributes from the second user input;
converting the second user input to a second instance of data defined by the plurality of second attributes;
compiling the second instance of data into the data set; and
transmitting the data set to a storage management tool via the network.

6. The turnover management system of claim 5, wherein the plurality of first attributes comprises a first specified workflow action, a first turnover identifier, a first timestamp, a first location, and a first role assignment.

7. The turnover management system of claim 6, wherein the plurality of second attributes comprises a second specified workflow action, the first turnover identifier, a second timestamp, the first location, and a second role assignment.

8. The turnover management system of claim 7, wherein the data set comprises a plurality of instances of data, each of the plurality of instances of data comprising the first turnover identifier.

9. A computer-based turnover management system comprising:
at least one computer readable non-transitory memory storing software instructions; and
at least one processor, coupled to the at least one memory, that performs operations upon execution of the software instructions, the operations comprising:
implementing a state-driven turnover workflow comprising a plurality of workflow actions;
enforcing state progression of the state-driven turnover workflow based upon satisfaction of at least one criterion;
initiating a first state upon satisfaction of a first criterion of the at least one criterion; terminating the first state and initiating a second state upon satisfaction of a second criterion of the at least one criterion; and
terminating the second state upon satisfaction of the second criterion.

10. The computer-based turnover management system of claim 9, wherein the at least one criterion comprises one or more user inputs.

11. The computer-based turnover management system of claim 10, wherein the one or more user inputs originates from one or more user devices.

12. The computer-based turnover management system of claim 10 wherein the one or more user inputs comprises at least two user inputs originating from one user device.

13. The computer-based turnover management system of claim 10 wherein the one or more user inputs comprises at least two user inputs originating from at least two user devices.

14. The computer-based turnover management system of claim 10 wherein the at least one criterion may consist of at least one user input, a passage of a predetermined period of time, or a combination thereof.

15. The computer-based turnover management system of claim 10, wherein the one or more user inputs may be selected from the group consisting of a binary user input, a freeform user input, a selection from a list, a photograph, or a combination thereof.

16. A method of managing turnover workflow comprising the steps:
(a) providing a turnover management system, said system comprising:
a turnover management tool having a processor and a memory storing a master program;
one or more user devices operatively coupled to the turnover management tool, the one or more user device comprising a display, a processor, and a memory storing a user application, the user application being subject to directives from the master program; and
a storage management tool comprising a storage device operatively coupled to the turnover management tool;
(b) initiating a workflow sequence displayed by the user application of the one or more user devices;
(c) recording at least one user input from the user application of the one or more user devices;
(d) transmitting the at least one user input to the master program of the turnover management tool;
(e) modifying, via the master program, the workflow sequence displayed by the user application of the one or more user devices based upon the at least one user input;
(f) recording the at least one user input as live data via the master program;
(g) terminating the workflow sequence;
(h) reclassifying the live data as historical data via the master program; and
(i) transmitting the historical data from the application management tool to the storage management tool.

17. The method of claim 16, wherein the turnover management system further comprises a network operatively coupled to the one or more user devices, the application management tool, and the storage management tool.

18. The method of claim 17, wherein said transmitting steps are performed via a network.

19. The method of claim 16, further comprising exporting the historical data to a spreadsheet.

20. The method of claim 16, wherein the at least one user input comprises at least two user inputs and the one or more user devices comprises at least two user devices.
